(19) United States
(12) Patent Application Publication
(10) Pub. No.: US 2015/0310169 A1
(43) Pub. Date: Oct. 29, 2015

(54) METABOLIC PROFILING IN TISSUE AND SERUM IS INDICATIVE OF TUMOR DIFFERENTIATION IN PROSTATE CANCER

(71) Applicants: Dana-Farber Cancer Institute, Inc., Boston, MA (US); The Brigham and Women's Hospital, Inc., Boston, MA (US)

(72) Inventors: Massimo Loda, Belmont, MA (US); Kathryn L. Penney, Arlington, MA (US); Svitlana Tyekucheva, Brighton, MA (US)

(21) Appl. No.: 14/441,716

(22) PCT Filed: Nov. 8, 2013
(86) PCT No.: PCT/US13/69153

§ 371 Date: May 8, 2015

Related U.S. Application Data
(60) Provisional application No. 61/724,410, filed on Nov. 9, 2012.
(60) Provisional application No. 61/783,980, filed on Mar. 14, 2013.

(51) Int. Cl. G06F 19/24; G06F 19/00; G01N 33/68

(52) U.S. Cl. CPC G06F 19/24; G01N 33/6848; G06F 19/3431; G01N 2570/00

(57) ABSTRACT

The invention provides methods and products to detect the presence of unidentified high grade prostate tumors in a subject with a Gleason score 7 prostate tumor. The method comprises obtaining a biological sample from a subject in need thereof, measuring a profile of metabolites in the biological sample, wherein the metabolites are differentially expressed in Gleason score 6 versus Gleason score 8 prostate tumors, and classifying the profile of the metabolites to assign a grade to the sample based on the profile of the metabolites.

### RELATED APPLICATION

This application claims the benefit under 35 U.S.C. §119(e) of U.S. provisional application serial No. 61/724,410, filed Nov. 9, 2012, and U.S. provisional application Ser. No. 61/783,980, filed Mar. 14, 2013, the contents of both of which are incorporated by reference herein in their entirety.

### FEDERALLY SPONSORED RESEARCH

This invention was made with Government support under National Institute of Health (NIH) Grant R01 CA131945. Accordingly, the Government has certain rights in this invention.

### BACKGROUND OF THE INVENTION

Clinicians and researchers are currently unable to distinguish at diagnosis with sufficient confidence men who with prostate cancer (CaP) have indolent disease from those who have aggressive disease. The most commonly used pathological grading system for prostate cancer is the Gleason Grading system, first developed by Donald F. Gleason in 1966. Gleason's system was (and remains) a unique pathological grading system created for prostate cancer since it is based entirely on the architectural pattern of the tumor without taking cytological features into account. Additionally, the system, rather than assigning the worst grade as the grade of the tumor, assigns a grade to the two most common grade patterns, the sum of which is reported as the Gleason score (Epstein et al. The 2005 International Society of Urological Pathology (ISUP) Consensus Conference on Gleason Grading of Prostatic Carcinoma. Am J Surg Pathol. 2005 Sepember;29(9):1228-42; Lotan et al. Clinical implications of changing definitions within the Gleason grading system. Nat Rev Urol. 2010 March;7(3):136-42). For example, if the most common tumor pattern was grade 3, and the next most common tumor pattern was grade 4, the Gleason Score would be 3+4=7.

Cancers with a higher Gleason score are more aggressive and have a worst prognosis. While tumors with Gleason score 6 may largely be indolent and tumors with Gleason score 8-10 may largely be potentially lethal, most men are graded as Gleason 7. Over 90,000 men are diagnosed with Gleason score 7 disease every year. Identifying predictors of outcome among patients with Gleason 7 disease is critical given that clinical outcomes among these patients are highly variable; many will die of their disease, while many others live a long and asymptomatic life. Albertsen et al. (20-year outcomes following conservative management of clinically localized prostate cancer. JAMA 2005;293(17):2095-101) followed for a median of 24 years a population of 767 men diagnosed with CaP in the pre-prostate specific antigen (PSA) era and treated conservatively (without surgery). CaP-specific mortality among Gleason 7 patients was 45% at 10 years. A modern, PSA-screened population is presumed to be more uniform than the population followed by Albertsen et al., since most men screened by PSA are diagnosed with localized disease. Yet even among patients determined to have localized disease, significant variability in outcome persists. A substantial number of Gleason 7 CaP patients are cured and a sizable minority develops lethal disease despite aggressive therapy. In a series of over 20,000 patients who underwent radical prostatectomy (RP) in the PSA era, CaP specific mortality was 23% for Gleason 4+3 disease at 20 years after diagnosis among men diagnosed in their 60s.

Among men with Gleason score 7, knowing the major Gleason grade helps with risk stratification. For men with Gleason score 7 disease with a major Gleason grade of 4 (4+3), the risk of lethal CaP may be significantly higher than among men with 3+4 disease (hazard ratio=3.1; 95% confidence interval (CI): 1.1-8.6; Stark et al. Gleason Score and Lethal Prostate Cancer: Does 3+4=4+3? J Clin Oncol 2009). However, whether 3+4 or 4+3, the vast majority of patients do not die from their disease. Attempts to further differentiate lethal from indolent disease beyond Gleason score have focused on developing molecular signatures. Such a signature, applied at diagnosis, could enhance the stratification of intermediate risk Gleason 7 patients and greatly impact treatment decisions

### SUMMARY OF THE INVENTION

It has been discovered, surprisingly, that metabolic profiles of biological samples, such as blood, are associated with the degree of differentiation in human prostate cancer, and can be used to detect unidentified high grade tumor, allowing the differentiation of aggressive from indolent tumors and enhancing risk prediction in Gleason 7 patients. Accordingly, in some aspects, the invention involves, supplementing Gleason score evaluation of a Gleason score 7 prostate tumor by obtaining a biological sample of a subject, measuring a profile of metabolites in the biological sample, wherein the metabolites are differentially expressed in Gleason score 6 versus Gleason score 8 prostate tumors, and classifying the profile of the metabolites to assign a supplemental Gleason grade to the sample based on the profile of the metabolites.

In some embodiments, the supplemental Gleason grade is 6 or 8. In some embodiments, the differentially expressed metabolites are selected using a criteria of false discovery rate <0.2.

In some embodiments, the differentially expressed metabolites are selected using a criteria of p-value <0.05. In some embodiments, the profile of metabolites is measured using one or more of mass spectroscopy, positron emission tomography or chromatography. The biological sample includes, but is not limited to blood, serum, urine, and tissue.

In some embodiments, the profile of metabolites is classified using a trained classifier. In some embodiments, the methods further comprise training a classifier to provide the trained classifier that distinguishes between Gleason grade 6 and 8 wherein the classifier is trained using a training set of samples comprising profiles of the metabolites that are differentially expressed in Gleason score 6 versus Gleason score 8 prostate tumors. In some embodiments, training the classifier comprises using a cross-validation technique. In some embodiments, the classifier is trained using the cross-validation technique until a correct Gleason grade of 6 or 8 is assigned to at least 75% of the samples in the training set of samples. In some embodiments, training the classifier comprises using linear discriminant analysis, logistic regression, regularized regression or support vector machines. In some embodiments, the methods further comprise training the classifier based on the classified profile of the biological sample. In some embodiments, the methods further comprise determining a confidence value for the Gleason grade assigned to the sample, and providing an indication of the confidence value and the Gleason grade assigned to the sample to a user.

According to some aspects of the invention, a method to detect the presence of high grade prostate tumors in a subject with a Gleason score 7 prostate tumor is provided. The method comprises obtaining a biological sample of a subject, measuring a profile of metabolites in the biological sample, wherein the metabolites are differentially expressed in Gleason score 6 versus Gleason score 8 prostate tumors, and analyzing the profile of the metabolites with at least one processor programmed to implement a specific prediction model to assign a Gleason grade to the sample based on the profile of the metabolites.

In some embodiments, the supplemental Gleason grade is 6 or 8. In some embodiments, the differentially expressed metabolites are selected using a criteria of false discovery rate <0.2. In some embodiments, the differentially expressed metabolites are selected using a criteria of p-value <0.05. In some embodiments, the profile of metabolites is measured using one or more of mass spectroscopy, positron emission tomography or chromatography. The biological sample includes, but is not limited to blood, serum, urine, and tissue.

In some embodiments, the specific prediction model comprises a trained classifier. In some embodiments, the method further comprises training a classifier to provide the trained classifier that distinguishes between Gleason grade 6 and 8 wherein the classifier is trained using a training set of samples comprising profiles of the metabolites that are differentially expressed in Gleason score 6 versus Gleason score 8 prostate tumors. In some embodiments, the classifier is trained using a cross-validation technique. In some embodiments, the classifier is trained using the cross-validation technique until a correct Gleason grade of 6 or 8 is assigned to at least 75% of the samples in the training set of samples. In some embodiments, the classifier is trained using linear discriminant analysis, logistic regression, regularized regression or support vector machines. In some embodiments, the method further comprises training the classifier based on the assigned metabolic profile of the biological sample. In some embodiments, the method further comprises determining a confidence value for the Gleason grade assigned to the sample, and providing an indication of the confidence value and the Gleason grade assigned to the sample to a user.

According to some aspects of the invention, a method to supplement Gleason score evaluation of a Gleason 7 prostate tumor is provided. The method comprises classifying, with at least one processor, a profile of a set of metabolites in a biological sample obtained from a subject with a Gleason score 7 prostate tumor to assign a Gleason grade to the sample based on the profile of metabolites, wherein metabolites in the set of metabolites are differentially expressed in Gleason score 6 versus Gleason score 8 prostate tumors.

In some embodiments, the supplemental Gleason grade is 6 or 8. In some embodiments, the differentially expressed metabolites are selected using a criteria of false discovery rate <0.2. In some embodiments, the differentially expressed metabolites are selected using a criteria of p-value <0.05. In some embodiments, the profile of metabolites is measured using one or more of mass spectroscopy, positron emission tomography or chromatography. The biological sample includes, but is not limited to blood, serum, urine, and tissue.

In some embodiments, the profile of metabolites is classified using a trained classifier. In some embodiments, the method further comprises training a classifier to provide the trained classifier that distinguishes between Gleason grade 6 and 8 wherein the classifier is trained using a training set of samples comprising profiles of the metabolites that are differentially expressed in Gleason score 6 versus Gleason score 8 prostate tumors. In some embodiments, training the classifier comprises using a cross-validation technique. In some embodiments, the classifier is trained using the cross-validation technique until a correct Gleason grade of 6 or 8 is assigned to at least 75% of the samples in the training set of samples. In some embodiments, training the classifier comprises using linear discriminant analysis, logistic regression, regularized regression or support vector machines. In some embodiments, the method further comprises training the classifier based on the classified profile of the biological sample. In some embodiments, classifying a profile of a set of metabolites in the biological sample comprises comparing at least some metabolites in the profile of the set of metabolites to a set of metabolites expressed in Gleason score 8 prostate tumors. In some embodiments, the method further comprises generating a report wherein the report indicates the assigned Gleason grade. In some embodiments, the method further comprises determining a confidence value for the Gleason grade assigned to the sample, and providing an indication of the confidence value and the Gleason grade assigned to the sample to a user.

According to some aspects of the invention, a method to train a classifier implemented using at least one processor is provided. The method comprises training, with at least one processor, a classifier to provide a trained classifier that distinguishes between Gleason score 6 and 8, wherein the classifier is trained using a training set of samples comprising profiles of the metabolites that are differentially expressed in Gleason score 6 versus Gleason score 8 prostate tumors.

In some embodiments, the differentially expressed metabolites are selected using a criteria of false discovery rate <0.2. In some embodiments, the differentially expressed metabolites are selected using a criteria of p-value <0.05. In some embodiments, the classifier is trained using a cross-validation technique. In some embodiments, the classifier is trained using the cross-validation technique until a correct Gleason grade of 6 or 8 is assigned to at least 75% of the sample in the training set of samples. In some embodiments, the classifier is trained using linear discriminant analysis, logistic regression, regularized regression or support vector machines.

According to some aspects of the invention, a computer-readable storage medium is provided. The medium is encoded with a plurality of instructions that, when executed by at least one processor, performs a method comprising classifying a profile of a set of metabolites in a biological sample obtained from a subject with a Gleason score 7 prostate tumor to assign a Gleason grade to the sample based on the profile of metabolites, wherein metabolites in the set of metabolites are differentially expressed in Gleason score 6 versus Gleason score 8 prostate tumors.

In some embodiments, the method further comprises determining a confidence value for the Gleason grade assigned to the sample, and providing an indication of the confidence value and the Gleason grade assigned to the sample to a user. In some embodiments, the method further comprises determining whether the confidence value is below a threshold value; and providing an indication that the confidence value is below the threshold value.

According to some aspects of the invention, a method to supplement Gleason score evaluation of a Gleason score 7 prostate tumor is provided. The method comprises performing an assay to measure an expression profile of metabolites in a biological sample obtained from a subject, and classifying, with at least one processor, the profile of the metabolites to assign a supplemental Gleason grade to the sample based on the profile of the metabolites.

According to some aspects of the invention, the method comprises performing an assay to measure an expression profile of metabolites in a biological sample obtained from a subject; and analyzing the profile of the metabolites with at least one processor programmed to implement a specific prediction model to assign a Gleason grade to the sample based on the profile of the metabolites.

According to some aspects of the invention, methods to diagnose prostate cancer in a subject, methods to determine the effectiveness of anti-cancer therapy and methods to monitor the progression or regression of prostate cancer are provided. The methods comprise performing an assay to measure an expression profile of metabolites in a biological sample obtained from a subject, and classifying the profile of the metabolites to determine the presence or absence of prostate cancer, the effectiveness of anti-cancer therapy or the progression or regression of prostate cancer. In some embodiments, the metabolites used in these methods are differentially expressed in prostate cancer patients before and after radical prostatectomy. In some embodiments, the differentially expressed metabolites are selected using a criteria of false discovery rate <0.2. In some embodiments, the differentially expressed metabolites are selected using a criteria of p-value<0.05. In some embodiments, the differentially expressed metabolites are selected from Table 6. In some embodiments, any subset of at least 5, at least 10, at least 15, at least 20 of the metabolites of Table 6 are used in the methods described herein. Examples of a subset of metabolites used in the methods described herein include, but are not limited to, the metabolites described in the column Cluster center: margarate (17:0), Cluster center: asparagine, Cluster center: nonadecanoate (19:0), Cluster center: cysteine or Cluster center: 4-androsten-3beta,17beta-diol disulfate 2. A non-limiting example of a subset metabolites used in the methods described herein is 1-arachidonoylglycerophosphoethanolamine, 2-hydroxydecanoic acid, 2-hydroxypalmitate, 3-hydroxydecanoate, 3-methoxytyrosine, dihomo-linoleate (20:2n6), gamma-glutamylglutamine, leucylglycine, margarate (17:0), palmitate (16:0), palmitoleate (16:1n7), phenylalanylleucine, tetradecanedioate, and undecanoate (11:0). In some embodiments, the metabolites are selected from Table 1, 3, 5 and/or 6.

The presence or absence of prostate cancer, the effectiveness of anti-cancer therapy or the progression or regression of prostate cancer is determined by classifying the profile of the metabolites. In some embodiments, classifying the profile of the metabolites comprises comparing the metabolic profile of the sample to an appropriate reference expression profile of the metabolites. An appropriate reference expression profile of the metabolites can be determined or can be a pre-existing reference profile. An appropriate reference expression profile includes the expression profile of the metabolites in a prostate cancer subject before and/or the expression profile of the metabolites in a prostate cancer subject after radical prostatectomy. A lack of a significant difference between the metabolic profile determined from the subject and the appropriate reference expression profile is indicative of the presence or absence of prostate cancer, the effectiveness of anti-cancer therapy or the progression or regression of prostate cancer.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

FIG. 1 shows examples of metabolites from the propanoate, beta-alanine, and pyrimidine metabolism pathways which differed between Gleason 3+3 and 4+4 tumors.

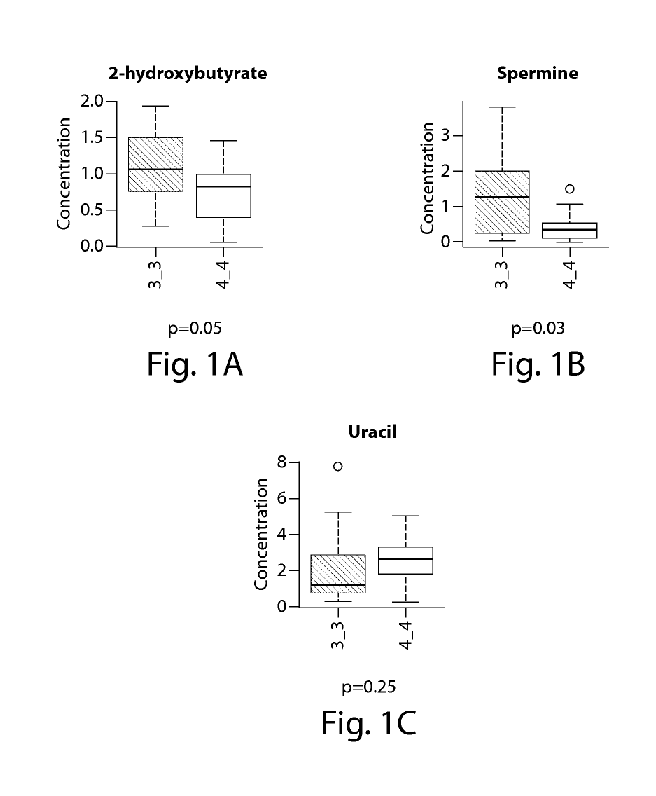

FIG. 2 shows examples of correlation between metabolite levels observed in tumors and corresponding sera samples.

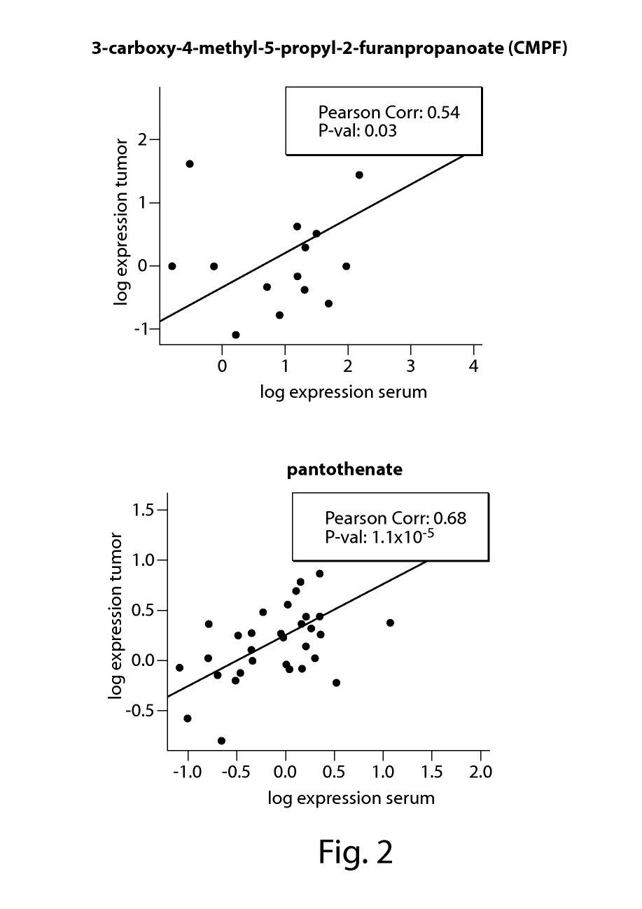

FIG. 3 is an illustrative implementation of a computer system.

FIG. 4 shows the unsupervised clustering with the significant tumor metabolites measured in all samples.

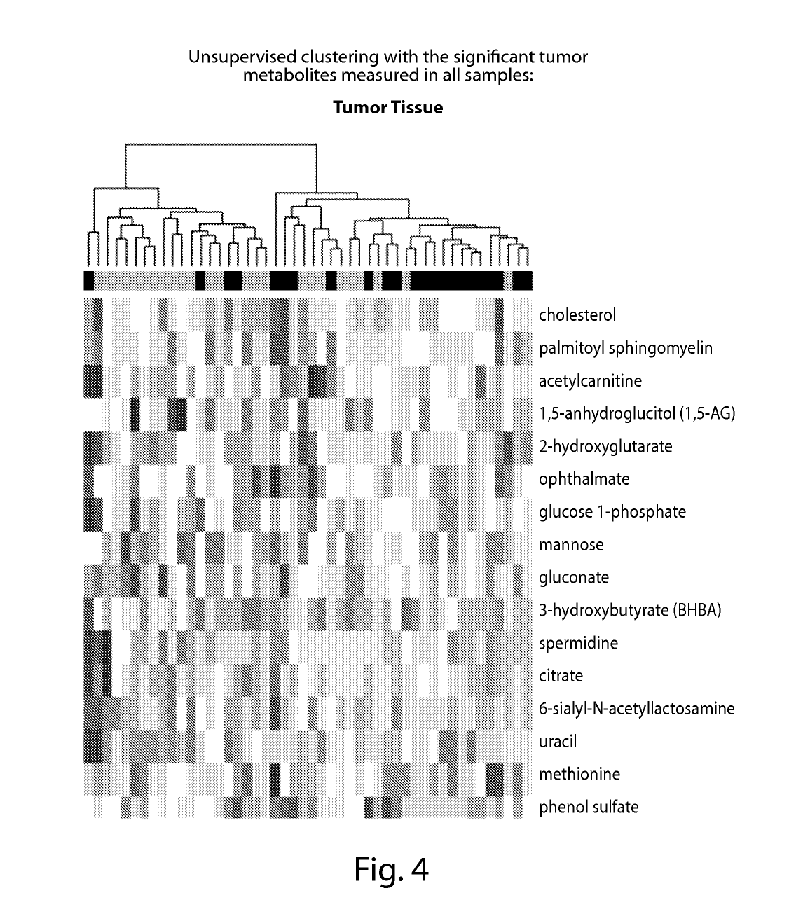

FIG. 5 shows the unsupervised clustering with the significant different metabolites in serum measured in all samples.

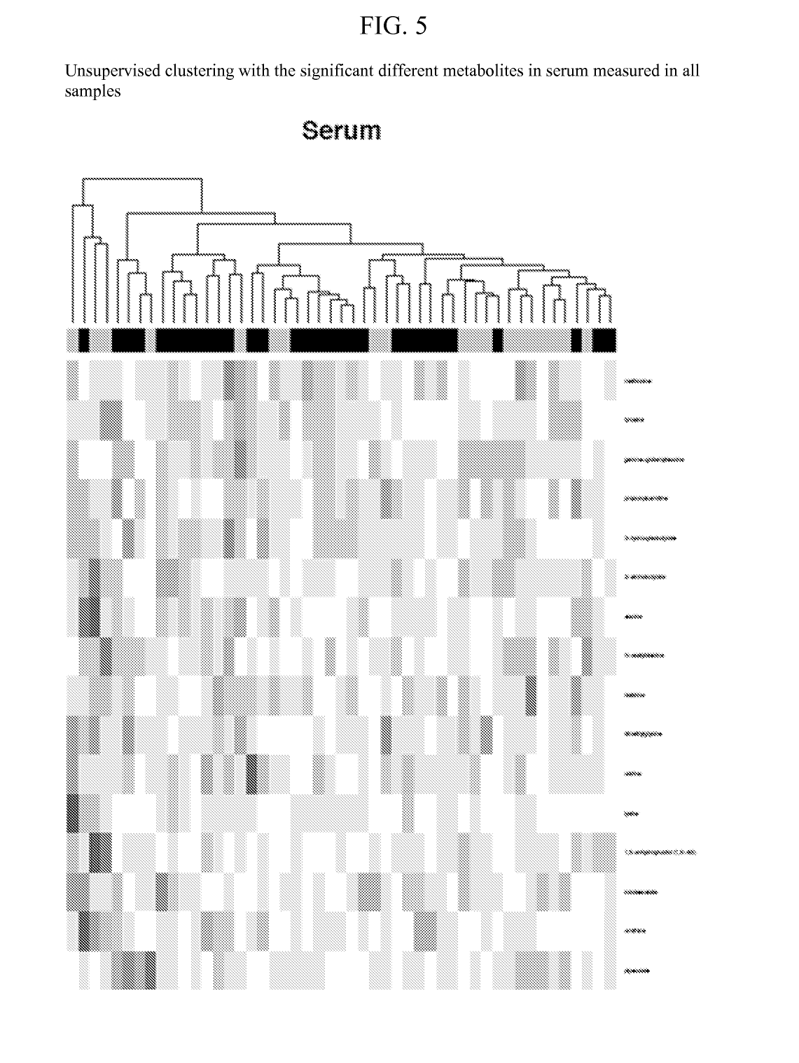

FIG. 6 shows clusters demonstrating the trends in average values of serum metabolites from before radical prostatectomy (Pre-RP) to two time points after surgery. Each figure is titled with the metabolite at the center of the cluster.

### DETAILED DESCRIPTION OF THE INVENTION

Currently, clinicians cannot identify with sufficient confidence Gleason 7 patients requiring aggressive therapy. This invention is based, at least in part, on the discovery that metabolic profiles are associated with the degree of differentiation in human prostate cancer. Metabolic assessment in biological samples, such as blood, can be used to detect unidentified high grade tumor, allowing the differentiation of aggressive from indolent tumors and enhancing risk prediction in Gleason 7 patients. Accordingly, aspects of the invention include methods to supplement Gleason score evaluation of a Gleason score 7 prostate tumor, methods to detect the presence of high grade prostate tumors in a subject with a Gleason score 7 prostate tumor, and methods to train a classifier implemented using a computer to provide a computer that uses the trained classifier to distinguish between Gleason score 6 and 8.

In some embodiments, the method described herein comprise obtaining a biological sample of a subject in need thereof; measuring a profile of metabolites in the biological sample, wherein the metabolites are differentially expressed in Gleason score 6 versus Gleason score 8 prostate tumors; and classifying the profile of the metabolites to assign a supplemental Gleason grade to the sample based on the profile of the metabolites.

Metabolites are small molecule compounds, such as substrates for enzymes of metabolic pathways, intermediates of such pathways or the products obtained by a metabolic pathway. Metabolic pathways are well known in the art, and include, for example, citric acid cycle, respiratory chain, glycolysis, gluconeogenesis, hexose monophosphate pathway, oxidative pentose phosphate pathway, production and β-oxidation of fatty acids, urea cycle, amino acid biosynthesis pathways, protein degradation pathways, amino acid degrading pathways, and biosynthesis or degradation of lipids, proteins, and nucleic acids. Accordingly, small molecule compound metabolites may be composed of the following classes of compounds: alcohols, alkanes, alkenes, alkines, aromatic compounds, ketones, aldehydes, carboxylic acids, esters, amines, imines, amides, cyanides, amino acids, peptides, thiols, thioesters, phosphate esters, sulfate esters, thioethers, sulfoxides, ethers, or combinations or derivatives of the aforementioned compounds.

Preferably, a metabolite has a molecular weight of 50 Da (Dalton) to 30,000 Da, most preferably less than 30,000 Da, less than 20,000 Da, less than 15,000 Da, less than 10,000 Da, less than 8,000 Da, less than 7,000 Da, less than 6,000 Da, less than 5,000 Da, less than 4,000 Da, less than 3,000 Da, less than 2,000 Da, less than 1,000 Da, less than 500 Da, less than 300 Da, less than 200 Da, less than 100 Da. Preferably, a metabolite has, however, a molecular weight of at least 50 Da. Most preferably, a metabolite in accordance with the present invention has a molecular weight of 50 Da up to 1,500 Da.

In some embodiments, at least some of the metabolites used in the methods described herein are differentially expressed in prostate tumors with Gleason score 6 versus prostate tumors with Gleason score 8. In some embodiments, the metabolites that are differentially expressed in prostate tumors with Gleason score 6 versus prostate tumors with Gleason score 8 are used in the methods described herein. By “differentially expressed” it means that the average expression of a metabolite in Gleason 6 subjects has a statistically significant difference from that in Gleason 8 subjects. For example, a significant difference that indicates differentially expressed metabolite may be detected when the expression level of the metabolite in a biological sample of a Gleason 6 subject is at least 1%, at least 5%, at least 10%, at least 25%, at least 50%, at least 100%, at least 250%, at least 500%, or at least 1000% higher, or lower, than that of a Gleason 8 subject. Similarly, a significant difference may be detected when the expression level of a metabolite in a biological sample of a Gleason 6 subject is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than that of a Gleason 8 subject. Significant differences may be identified by using an appropriate statistical test. Tests for statistical significance are well known in the art and are exemplified in Applied Statistics for Engineers and Scientists by Petruccelli, Chen and Nandram 1999 Reprint Ed. In some embodiments, the differentially expressed metabolites are selected using a criteria of false discovery rate <0.2. In some embodiments, the differentially expressed metabolites are selected using a criteria of p-value <0.05. P-value looks at the average concentration of the metabolite in the two groups and tells you how likely is it that the difference in the concentration between the two groups occurs by chance.

In some embodiments, the metabolites used in the methods described herein are selected from Table 2. In some embodiments, any subset of at least 5, at least 10, at least 15, at least 20, at least 25, at least 30 of the metabolites of Table 2 are used in the methods described herein. Examples of a subset of metabolites used in the methods described herein include, but are not limited to, the first 5, 10, 15, 20, 25, or 30 metabolites or the last 5, 10, 15, 20, 25, or 30 metabolites or any combination of 5, 10, 15, 20, 25, or 30 metabolites of Table 2. In some embodiments, at least 5, at least 10, at least 15, at least 20, at least 25, or at least 30 of the metabolites of Table 2 with the lowest p-value are used in the methods described herein. A non-limiting example of a subset of at least 5 metabolites used in the methods described herein is spermine, spermidine, citrate, N-acetylputrescine, and palmitoyl sphingomyelin.

In some embodiments, the metabolites used in the methods described herein are selected from Table 3. In some embodiments, any subset of at least 5, at least 10, at least 15, at least 20 of the metabolites of Table 3 are used in the methods described herein. Examples of a subset of metabolites used in the methods described herein include, but are not limited to, the first 5, 10, 15, or 20 metabolites or the last 5, 10, 15, or 20 metabolites or any combination of 5, 10, 15, or 20 metabolites of Table 3. In some embodiments, at least 5, at least 10, at least 15, or at least 20 of the metabolites of Table 3 with the lowest p-value are used in the methods described herein. A non-limiting example of a subset of at least 5 metabolites used in the methods described herein is N-acetylserine, beta-alanine, proprionylcarnitine, N-acetylalanine and pyrophosphate.

In some embodiments, the metabolites used in the methods described herein are selected from Table 5. In some embodiments, any subset of at least 5, at least 10, at least 15, at least 20 of the metabolites of Table 5 are used in the methods described herein. Examples of a subset of metabolites used in the methods described herein include, but are not limited to, the first 5, 10, 15, or 20 metabolites or the last 5, 10, 15, or 20 metabolites or any combination of 5, 10, 15, or 20 metabolites of Table 5. In some embodiments, at least 5, at least 10, at least 15, or at least 20 of the metabolites of Table 5 with the lowest p-value are used in the methods described herein. A non-limiting example of a subset of at least 5 metabolites used in the methods described herein is 1-palmitoylglycerophosphoethanolamine, creatine, methyl-alpha-glucopyranoside, adenosine, ethanolamine, taurine, guanosine 5′-monophosphate (GMP), methylphosphate, and guanosine.

In some embodiments, a combination of metabolites selected from Table 2, Table 3, Table 5 and Table 6 are used in the methods described herein. In some embodiments, a combination of metabolites selected from Table 2, Table 3 and Table 5 are used in the methods described herein.

As used herein, a “subject” refers to any male mammal, including humans and non-humans, such as primates. Typically the subject is a human, and has been diagnosed or is suspected of having a prostate tumor with Gleason score 7. In some embodiments, the subject may be diagnosed as having prostate tumor with Gleason score 7 using one or more of the following tests: digital rectal exam (DRE), prostate imaging, biopsy with Gleason grading evaluation, presence of tumor markers such as PSA and prostate cancer staging (Lumen et al. Screening and early diagnosis of prostate cancer: an update. Acta Clin Belg. 2012 July-August; 67(4):270-5).

A subject suspected of having Gleason 7 prostate tumor may be a subject having one or more clinical symptoms of prostate tumor. A variety of clinical symptoms of prostate cancer are known in the art. Examples of such symptoms include, but are not limited to, frequent urination, nocturia (increased urination at night), difficulty starting and maintaining a steady stream of urine, hematuria (blood in the urine), dysuria (painful urination) and bone pain.

The term “biological sample” refers to a sample derived from a subject, e.g., a patient. Non-limiting examples of the biological sample include blood, serum, urine, and tissue. Obtaining a biological sample of a subject means taking possession of a biological sample of the subject. Obtaining a biological sample from a subject means removing a biological sample from the subject. Therefore, the person obtaining a biological sample of a subject and measuring a profile of metabolites in the biological sample does not necessarily obtain the biological sample from the subject. In some embodiments, the biological sample may be removed from the subject by a medical practitioner (e.g., a doctor, nurse, or a clinical laboratory practitioner), and then provided to the person measuring a profile of metabolites. The biological sample may be provided to the person measuring a profile of metabolites by the subject or by a medical practitioner (e.g., a doctor, nurse, or a clinical laboratory practitioner). In some embodiments, the person measuring a profile of metabolites obtains a biological sample from the subject by removing the sample from the subject.

It is to be understood that a biological sample may be processed in any appropriate manner to facilitate measuring expression levels of metabolic profiles. For example, biochemical, mechanical and/or thermal processing methods may be appropriately used to isolate a biomolecule of interest from a biological sample. The expression levels of the metabolites may also be determined in a biological sample directly. The expression levels of the metabolites may be measured by performing an assay, such as but not limited to, mass spectroscopy, positron emission tomography, gas chromatography (GC-MS) or HPLC liquid chromatography (LC-MS). Other appropriate methods for determining levels of metabolites will be apparent to the skilled artisan.

The methods disclosed herein typically comprises measuring and classifying the expression profiles of differentially expressed metabolites. In some embodiments, at least 5, at least 10, at least 25, at least 50, at least 75, at least 100, at least 125, at least 150, at least 175, at least 200, at least 225, at least 250, at least 500, at least 750, at least 1000 or at least 1500 differentially expressed metabolites are measured and classified to assign a grade to the sample.

“Assign a supplemental grade”, “assign a Gleason grade” or “assign a grade” means identifying with at least one processor the sample as having a metabolite expression profile that is similar to or characteristic of a Gleason score 6 or Gleason score 8 tumor. In some embodiments, the sample is assigned by the processor a Gleason score of 6 or 8 based on the profile of metabolites. The assigned grade along with additional information such the results of a PSA test and prostate imaging, is used to determine which subject requires radical prostatectomy. A report summarizing the results of the analysis, i.e. the assigned grade of the sample and any other information pertaining to the analysis could optionally be generated as part of the analysis (which may be interchangeably referred to herein as “providing” a report, “producing” a report, or “generating” a report). Examples of reports may include, but are not limited to, reports in paper (such as computer-generated printouts of test results) or equivalent formats and reports stored on computer readable medium (such as a CD, computer hard drive, or computer network server, etc.). Reports, particularly those stored on computer readable medium, can be part of a database (such as a database of patient records, which may be a “secure database” that has security features that limit access to the report, such as to allow only the patient and the patient's medical practitioners to view the report, for example). In addition to, or as an alternative to, generating a tangible report, reports can also be displayed on a computer screen (or the display of another electronic device or instrument).

A report can further be transmitted, communicated or reported (these terms may be used herein interchangeably), such as to the individual who was tested, a medical practitioner (e.g., a doctor, nurse, clinical laboratory practitioner, genetic counselor, etc.), a healthcare organization, a clinical laboratory, and/or any other party intended to view or possess the report. The act of ‘transmitting’ or ‘communicating’ a report can be by any means known in the art, based on the form of the report, and includes both oral and non-oral transmission. Furthermore, “transmitting” or “communicating” a report can include delivering a report (“pushing”) and/or retrieving (“pulling”) a report. For example, non-oral reports can be transmitted/communicated by such means as being physically transferred between parties (such as for reports in paper format), such as by being physically delivered from one party to another, or by being transmitted electronically or in signal form (e.g., via e-mail or over the internet, by facsimile, and/or by any wired or wireless communication methods known in the art), such as by being retrieved from a database stored on a computer network server, etc.

The grade of the biological sample isolated from a subject is assigned by classifying the profile of the metabolites of the sample. In some embodiments, classifying the profile of the metabolites comprises comparing the metabolic profile of the sample to an appropriate reference expression profile of the metabolites. An appropriate reference expression profile of the metabolites can be determined or can be a pre-existing reference profile. An appropriate reference expression profile includes the expression profile of the metabolites in a Gleason 6 subject and/or the expression profile of the metabolites in a Gleason 8 subject. A lack of a significant difference between the metabolic profile determined from the subject and the appropriate reference expression profile is indicative of the grade of the sample.

In some embodiments, the methods described herein may involve building a prediction model, which may also be referred to as a classifier or predictor, that can be used to classify the disease status of an individual. Thus, aspects of the invention involve methods to detect the presence of high grade tumors in a subject with a Gleason score 7 prostate tumor by using at least one processor programmed to implement the classifier or predictor to analyze or classify the profile of metabolites in a sample isolated from the subject. The classifier or predictor assigns a grade to the sample isolated from a subject based on the profile of the metabolites. Typically the model is built using samples for which the classification (grade) has already been ascertained. Once the model is built/trained, it may be applied to metabolic profiles obtained from a biological sample in order to classify and assign a grade to the sample isolated from the subject. Thus, the methods may involve applying a trained classifier to the metabolic profiles, such that the trained classifier assigns a grade to the sample based on the expression levels. The subject may be further diagnosed, e.g., by a health care provider, based on the assigned grade.

A variety of prediction models known in the art may be used as the classifier or predictor. For example, the classifier may be established using logistic regression, partial least squares, linear discriminant analysis, regularized regression, quadratic discriminant analysis, neural network, naïve Bayes, C4.5 decision tree, k-nearest neighbor, random forest, and support vector machine.

The classifier may be trained on a data set comprising profiles of the metabolites that are differentially expressed in individuals identified as having Gleason score 6 versus Gleason score 8 prostate tumors. For example, the classifier may be trained on a data set comprising metabolic profiles in samples obtained from a plurality of individuals identified as having Gleason score 6 or 8 prostate tumors based on Gleason grading system. The training set may also comprise metabolic profiles of control individuals identified as not having prostate tumor. As will be appreciated by the skilled artisan, the population of individuals of the training data set may have a variety of characteristics by design, e.g., the characteristics of the population may depend on the characteristics of the individuals for whom diagnostic methods that use the classifier may be useful. For example, the interquartile range of ages of a population in the training data set may be from about 35 years old to about 85 years old, or more.

A class prediction strength can also be measured to determine the degree of confidence with which the model classifies a biological sample. The prediction strength conveys the degree of confidence of the classification of the sample and evaluates when a sample cannot be classified. There may be instances in which a sample is tested, but does not belong, or cannot be reliably assigned to, a particular class. This is done by utilizing a threshold in which a sample which scores above or below the determined threshold is not a sample that can be classified (e.g., a “no call”). In such instances, the classifier may provide an indication that the confidence value is below the threshold value. In some embodiments, the sample is then manually classified to assign a grade to the sample.

Once a classifier is developed, the validity of the classifier can be tested using methods known in the art. One way to test the validity of the model is by cross-validation of the dataset. In some embodiments, the cross-validation technique comprises a leave-one-out cross-validation. To perform leave-one-out cross-validation, one, or a subset, of the samples is eliminated and the classifier is built, as described above, without the eliminated sample, forming a “cross-validation model.” The eliminated sample is then classified according to the classifier, as described herein. This process is done with all the samples, or subsets, of the initial dataset and an error rate is determined. The accuracy of the classifier is then assessed. This classifier classifies samples to be tested with high accuracy for classes that are known, or classes have been previously ascertained. Another way to validate the classifier is to apply the model to an independent data set, such as a new biological sample of a subject having prostate tumor with an unknown grade. Other appropriate validation methods will be apparent to the skilled artisan. In some embodiments, the classifier is trained using the cross-validation technique until a correct Gleason grade of 6 or 8 is assigned to at least 75% of the samples in the training set of samples. In some embodiments, the classifier is trained until at least 80%, at least 85%, at least 90%, at least 95%, at least 99% or 100% of the samples in the training set are correctly assigned grade.

As will be appreciated by the skilled artisan, the strength of the classifier may be assessed by a variety of parameters including, but not limited to, the accuracy, sensitivity, specificity and area under the receiver operation characteristic curve. Methods for computing accuracy, sensitivity and specificity are known in the art. The classifier may have an accuracy of at least 60%, at least 65%, at least 70%, at least 75%, at least 80%, at least 85%, at least 90%, at least 95%, at least 99%, or more. The classifier may have an accuracy score in a range of about 60% to 70%, 70% to 80%, 80% to 90%, or 90% to 100%. The classifier may have a sensitivity score of at least 60%, at least 65%, at least 70%, at least 75%, at least 80%, at least 85%, at least 90%, at least 95%, at least 99%, or more. The classifier may have a sensitivity score in a range of about 60% to 70%, 70% to 80%, 80% to 90%, or 90% to 100%. The classifier may have a specificity score of at least 60%, at least 65%, at least 70%, at least 75%, at least 80%, at least 85%, at least 90%, at least 95%, at least 99%, or more. The classifier may have a specificity score in a range of about 60% to 70%, 70% to 80%, 80% to 90%, or 90% to 100%.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the present invention comprises at least one non-transitory computer-readable storage medium (e.g., a computer memory, a USB drive, a flash memory, a compact disk, a tape, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

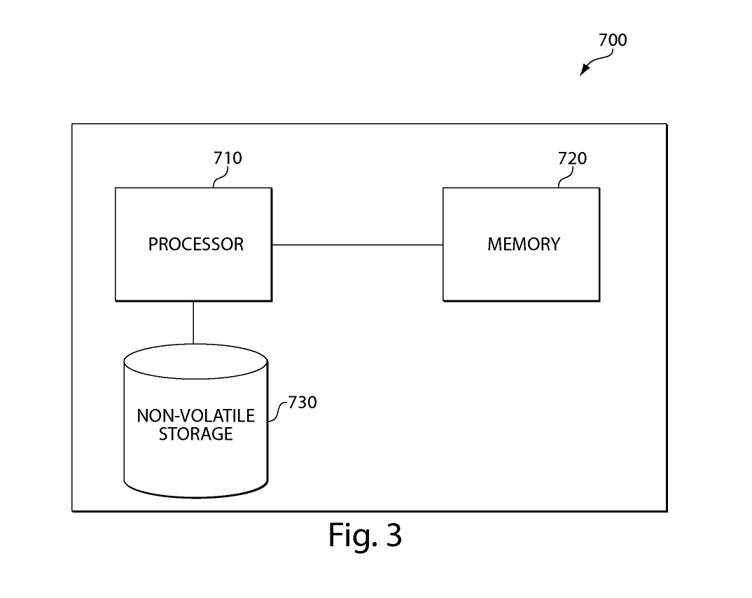

An illustrative implementation of a computer system 700 that may be used in connection with any of the embodiments of the invention described herein is shown in FIG. 3. The computer system 700 may include one or more processors 710 and one or more computer-readable tangible non-transitory storage media (e.g., memory 720, one or more non-volatile storage media 730, or any other suitable storage device). The processor 710 may control writing data to and reading data from the memory 720 and the non-volatile storage device 730 in any suitable manner, as the aspects of the present invention described herein are not limited in this respect. To perform any of the functionality described herein, the processor 710 may execute one or more instructions stored in one or more computer-readable storage media (e.g., the memory 720), which may serve as tangible non-transitory computer-readable storage media storing instructions for execution by the processor 710.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1

### Methods:

Metabolic profiling (“metabolomics”) data for both prostate tumor specimens and pre-radical prostatectomy serum specimens are generated by Metabolon, Inc (Durham, N.C.). The levels of metabolites are measured on GC/MS and LC/MS/MS platforms. Metabolic profiling of serial serum samples taken before radical prostatectomy and over time after surgery are performed to insure that metabolites examined in sera are prostate or tumor-specific.

### Data Preparation

If a majority (>50%) of measurements within each phenotype group are missing, the data are considered sparse and not analyzed. Missing values for non-sparse markers will be imputed with minimum values observed for that compound, under the assumption that a missing value occurs because the level is below the level of detection (LOD) of the instruments (i.e. is non-random). For each metabolite the observed levels are normalized to the median values of the run day, to correct for instrument inter-day tuning differences.

### Statistical Analysis

Both tumor metabolite levels and their fold-changes with respect to the levels in paired normal prostate tissue are considered which reduces between patient variability. Levels of each metabolite or its fold-changes are then compared across Gleason score categories with the appropriate parametric (t-test) or non-parametric two-sample (Mann-Whitney) test as determined by checking distributional assumptions for each test. Significantly different metabolites (at the 0.2 FDR threshold) are used to build a discrimination model. Levels of the significant metabolites will be used in Linear Discriminant Analysis (LDA) to build a hyperplane separating the two groups (samples of Gleason 6 and Gleason 8 tumors). Parameters of the classifier will be tuned using cross-validation.

The LDA classifier is applied to the metabolomic profiles of men with Gleason score 7 in order to determine its accuracy in predicting lethal events for those patients.

To analyze metabolomic profiles from the serial sera samples longitudinal generalized linear modeling is employed. The fasting time before blood draw is included in the model, to control for potential differences due to fasting status. In order for a metabolite to be considered a true potential marker of Gleason score, it must have a consistently high fold-change with respect to the pre-radical prostatectomy baseline across all samples once the tumor and prostate are removed. This exercise identification of metabolites that are truly secreted by the tumor tissue or the prostate (FDR of 0.2 will be used as a significance threshold).

For serum metabolites, measurement and analysis will be the same as that described above for the tumor specimens. The focus of the study are the prostate-specific secreted metabolites as determined by the analysis of serial sera samples. As described above, two-sample tests are used to determine metabolites significantly different between sera samples of Gleason 6 and 8 patients, and those metabolites are used to construct a LDA classifier. This classifier is applied to metabolomic profiles from blood samples taken at the time of diagnosis in an independent cohort of patients with biopsy Gleason of 6 or 7 and known radical prostatectomy Gleason scores (approximately half upgraded) to evaluate its utility in predicting the radical prostatectomy Gleason score upgrade.

This study develops:

1. A metabolomic signature (classifier) that can be applied to prostate tumor tissue to improve prediction of lethal outcome among men with intermediate Gleason score 7 disease.

2. A metabolic test that can be carried out in serum from prostate cancer patients after diagnostic biopsy but before they have undergone aggressive treatment. This serum test could detect if higher grade tumor is present in the prostate but was not detected by the random biopsy. If men and their physicians choose active surveillance, this test can also help monitor patients to determine if higher grade tumors develop.

Metabolic profiling was performed on Gleason score 6 (n=24) and Gleason score 8 (n=9) radical prostatectomy tumor specimens and 31 matched post-diagnostic sera samples from DF/HCC Prostate Cancer SPORE Cohort patients. Samples were prepared for analysis by Metabolon with their standard solvent extraction method to recover small molecules. Metabolites were identified and quantified by gas and liquid chromatography and mass spectrometry. The metabolite levels were compared across tumors with Gleason score 6 and Gleason score 8 with two-sample t-tests. Pearson correlation coefficients were calculated for metabolite levels in serum and levels in tumor tissue.

### Results:

A 157 gene mRNA signature that distinguished high from low Gleason score and predicted lethal disease in men with clinically heterogeneous Gleason 7 was previously identified. In the tumor tissue, 307 metabolites were identified. Consistent with mRNA data, biochemicals in specified metabolic pathways exhibited differential abundance comparing Gleason score 6 to Gleason score 8 (selected examples: 2-hydroxybutyrate p=0.05; spermine p=0.03). Of 178 metabolites present in both serum and tumor, 10 had Pearson correlation coefficients >0.60. Pantothenate, directly downstream of beta-alanine, had a correlation coefficient of 0.68 (p=1.1×10-5). 3-carboxy-4-methyl 5-propyl-2-furanpropanoate, in the propanoate pathway, had a correlation coefficient of 0.54 (p=0.03).

TABLE 1
Gene pathways enriched in high grade or low grade tumors,
based on GSEA using Molecular Signature Databases from the
Broad Institute (FDR < 0.1), included metabolic pathways.

| Pathways enriched in high-grade | Pathways enriched in low-grade |
|---|---|
| CELL_CYCLE_KEGG | PROPANOATE_METABOLISM |
| CELL_CYCLE | HSA_PROPANOATE_METABOLISM |
| G1_TO_S_CELL_CYCLE_REACTOME | BETA_ALANINE_METABOLISM |
| DNA_REPLICATION_REACTOME | |
| PTDINSPATHWAY | |
| GSK3PATHWAY | |
| ST_GA13_PATHWAY | |
| PYRIMIDINE_METABOLISM | |
| HSA_PYRIMIDINE_METABOLISM | |
| ONE_CARBON_POOL_BY_FOLATE | |
| HSA_ONE_CARBON_POOL_BY_FOLATE | |
| MRNA_PROCESSING_REACTOME | |
| EPOPATHWAY | |
| NOTCH_SIGNALING_PATHWAY | |
| UBIQUITIN_MEDIATED_PROTEOLYSIS | |

### Example 2

Additional metabolic profiling was performed on Gleason score 6 (n=23) and Gleason score 8 (n=25) radical prostatectomy tumor specimens and on Gleason score 6 (n=30) and Gleason score 8 (n=19) serum samples. Majority of the cases for serum and tumor data are paired. Samples were prepared for analysis by Metabolon with their standard solvent extraction method to recover small molecules. Metabolites were identified and quantified by gas and liquid chromatography and mass spectrometry. The metabolite levels were compared across tumors with Gleason score 6 and Gleason score 8 with two-sample t-tests.

Several metabolites were found to be present in significantly different amounts in Gleason score 6 and Gleason score 8 patients. Results from these new samples are as follows:

TABLE 2
Metabolites significantly different between
high and low Gleason in tumor tissue:

| Metabolite | p-value |
|---|---|
| spermine | 0.000371522 |
| spermidine | 0.000483048 |
| citrate | 0.000526774 |
| N-acetylputrescine | 0.000778788 |
| palmitoyl sphingomyelin | 0.001531554 |
| alpha-ketoglutarate | 0.001993008 |
| 6-sialyl-N-acetyllactosamine | 0.002419546 |
| putrescine | 0.002836745 |
| cis-aconitate | 0.003346064 |
| glucose 1-phosphate | 0.003485425 |
| mannose | 0.003485425 |
| adenosine 5′diphosphoribose | 0.004295425 |
| adenosine 5′-triphosphate (ATP) | 0.006072343 |
| gluconate | 0.007408483 |
| 2-hydroxyglutarate | 0.00776042 |
| decanoylcarnitine | 0.008219927 |
| glucose-6-phosphate (G6P) | 0.008673201 |
| acetylcarnitine | 0.009595198 |
| uracil | 0.010878669 |
| cholesterol | 0.011577215 |
| xanthine | 0.014526872 |
| 1,5-anhydroglucitol (1,5-AG) | 0.017649469 |
| N-acetylneuraminate | 0.018551703 |
| 2-palmitoylglycerol (2-monopalmitin) | 0.019504626 |
| methionine | 0.023506443 |
| phenol sulfate | 0.029312268 |
| 3-hydroxybutyrate (BHBA) | 0.032641521 |
| fructose-6-phosphate | 0.036010044 |
| 4-androsten-3beta,17beta-diol disulfate 1* | 0.040609456 |
| ophthalmate | 0.041035082 |
| pyroglutamylvaline | 0.043018873 |

TABLE 3
Metabolites significantly different between
high and low Gleason in serum:

| Metabolite | p-value |
|---|---|
| N-acetylserine | 0.001143932 |
| beta-alanine | 0.010724582 |
| propionylcarnitine | 0.013192646 |
| N-acetylalanine | 0.013410757 |
| pyrophosphate (PPi) | 0.017731021 |
| pipecolate | 0.017762331 |
| tyrosine | 0.022337539 |
| arginine | 0.023047306 |
| indoleacetate | 0.024978277 |
| 3-hydroxyisobutyrate | 0.027878836 |
| gamma-CEHC | 0.02890912 |
| 1,5-anhydroglucitol (1,5-AG) | 0.029432548 |
| lysine | 0.029432548 |
| uridine | 0.029432548 |
| gamma-glutamylleucine | 0.030397677 |
| ornithine | 0.03105851 |
| alanine | 0.032835462 |
| histidine | 0.034537347 |
| methionine | 0.038336115 |
| 2-aminobutyrate | 0.040362149 |
| dimethylglycine | 0.044681211 |

A Linear discriminant analysis model was trained on the available data and its ability to distinguish Gleason 6 and Gleason 8 tumors based on the observed levels of the metabolites was assessed. Misclassification rates quantify this ability. Resubstitution rates evaluate the performance of the model on the data at hand, and rates from cross-validation allow to estimate misclassification rates when the model is applied to new data. The predictive ability of the model was high for both tumor and serum (Table 4), and can further enhanced by tuning the model's sensitivity to recognize Gleason 8 patterns (from the clinical perspective, misclassifying generally less aggressive Gleason 6 tumors as more aggressive Gleason 8 is less important, than to erroneously classify a Gleason 8 as a Gleason 6).

TABLE 4
LDA misclassification rates: These are the percentages of
Gleason misclassified using two different statistical methods
with the tumor tissue and serum metabolite data.

| | Tissue | Serum |
|---|---|---|
| Resubstitution | 0.0833 | 0.163 |
| Cross- | 0.25 | 0.367 |
| validation | | |

TABLE 5
Analysis of 35 patients with G8 and 38 with G6 tumors. P values
are based on Wilcoxon test between high and low grade.

| | |
|---|---|
| adenine | 0.001883704 |
| 1-palmitoylglycerophosphoethanolamine | 0.003613418 |
| creatine | 0.005483679 |
| methyl-alpha-glucopyranoside | 0.007379523 |
| adenosine | 0.013395596 |
| ethanolamine | 0.013801381 |
| taurine | 0.020683537 |
| guanosine 5′-monophosphate (GMP) | 0.022144522 |
| methylphosphate | 0.024008926 |
| guanosine | 0.025713779 |
| glycerophosphorylcholine (GPC) | 0.026455089 |
| glutathione, reduced (GSH) | 0.018448492 |
| butyrylcarnitine | 0.031152565 |
| glycerophosphoethanolamine | 0.031966504 |
| myo-inositol | 0.034863671 |
| oleoylcarnitine | 0.040156363 |
| kynurenine | 0.042179044 |
| 2-oleoylglycerophosphoethanolamine* | 0.042196915 |
| agmatine | 0.046131481 |
| nicotinamide | 0.048093796 |

### Example 3

Further studies were conducted to characterize prostate and prostate cancer-specific metabolites in serum. An additional study comparing serum samples from three time points was performed: days before radical prostatectomy (surgical removal of the prostate), within weeks after surgery, and within two years after surgery.

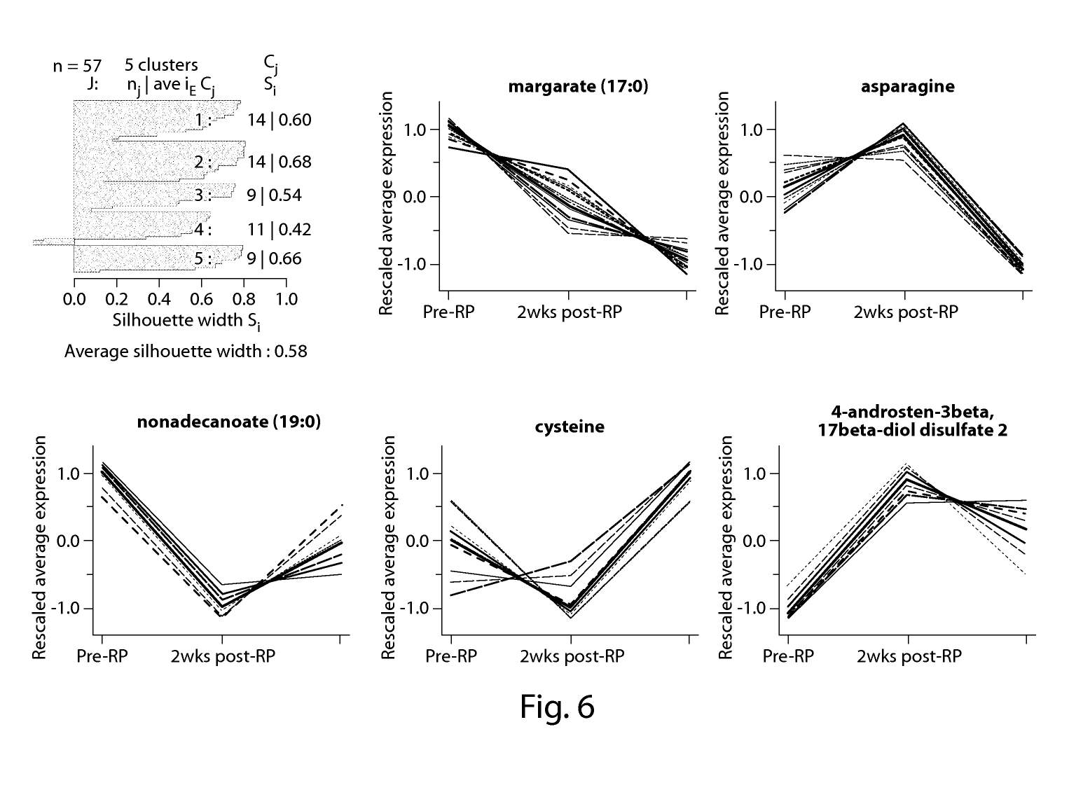

Metabolomic profiling was generated for 27 prostate cancer patients on serum samples from these three time points. Of all of the metabolites measured, 57 had high levels of variability. The data were normalized and an average of all patients' values was calculated for each metabolite at each time point. A clustering analysis was performed; the 57 metabolites cluster well into five groups (Table 6; FIG. 6). The trends in values across the time points demonstrates candidates for prostate-specific metabolites. In FIG. 6, specifically the “margarate (17:0)” cluster shows a decrease from before to after surgery, which means the metabolites in this cluster are excellent candidates as prostate-specific biomarkers.

TABLE 6

| | | | | Cluster center: 4- |
|---|---|---|---|---|
| | | Cluster center: | | androsten- |
| Cluster center: | Cluster center: | nonadecanoate | Cluster center: | 3beta,17beta-diol |
| margarate (17:0) | asparagine | (19:0) | cysteine | disulfate 2* |
| 1- | 1- | 1- | 15- | 4-androsten- |
| arachidonoyl- | linoleoyl | oleoylglycero- | methylpalmitate | 3beta,17beta-diol |
| glycerophospho- | glycerol (1- | phosphoethanolamine | (isobar with 2- | disulfate 2* |
| ethanolamine* | monolinolein) | | methylpalmitate) | |
| 2- | 1- | 10- | cortisol | decanoylcarnitine |
| hydroxydecanoic | stearoylglycero- | heptadecenoate | | |
| acid | phosphoethanol- | (17:1n7) | | |
| | amine | | | |
| 2- | asparagine | 2- | cysteine | HWESASXX* |
| hydroxypalmitate | | oleoylglycero- | | |
| | | phosphoethanolamine* | | |
| 3- | azelate | alpha- | cystine | N-acetylalanine |
| hydroxydecanoate | (nonanedioate) | glutamyltyrosine | | |
| 3- | dihomo- | chiro-inositol | deoxycarnitine | phenylalanine |
| methoxytyrosine | linolenate | | | |
| | (20:3n3 or n6) | | | |
| dihomo-linoleate | gamma- | mannose | glutamine | pro-hydroxy-pro |
| (20:2n6) | glutamylisoleucine* | | | |
| gamma- | gamma- | myristate (14:0) | guanosine | S-methylcysteine |
| glutamylglutamine | glutamylvaline | | | |
| leucylglycine | glycerate | nonadecanoate | palmitoyl | stearoyl |
| | | (19:0) | sphingomyelin | sphingomyelin |
| margarate (17:0) | glycylproline | pyruvate | taurochenodeoxy- | threonine |
| | | | cholate | |
| palmitate (16:0) | leucylleucine | | taurolithocholate | |
| | | | 3-sulfate | |
| palmitoleate | proline | | xylose | |
| (16:1n7) | | | | |
| phenylalanylleucine | sebacate | | | |
| | (decanedioate) | | | |
| tetradecanedioate | threonylphenyl | | | |
| | alanine | | | |
| undecanoate | undecanedioate | | | |
| (11:0) | | | | |

The foregoing written specification is considered to be sufficient to enable one skilled in the art to practice the invention. The present invention is not to be limited in scope by examples provided, since the examples are intended as a single illustration of one or more aspects of the invention and other functionally equivalent embodiments are within the scope of the invention.

Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method to supplement Gleason score evaluation of a Gleason score 7 prostate tumor, the method comprising:
obtaining a biological sample of a subject in need thereof;
measuring a profile of metabolites in the biological sample, wherein the metabolites are differentially expressed in Gleason score 6 versus Gleason score 8 prostate tumors; and
classifying the profile of the metabolites to assign a supplemental Gleason grade to the sample based on the profile of the metabolites.

2. The method of claim 1, wherein the supplemental Gleason grade is 6 or 8.

3. The method of claim 1, wherein the differentially expressed metabolites are selected using a criteria of false discovery rate <0.2.

4. (canceled)

5. The method of claim 1, wherein the profile of metabolites is classified using a trained classifier.

6. The method of claim 5, further comprising:
training a classifier to provide the trained classifier that distinguishes between Gleason grade 6 and 8 wherein:
the classifier is trained using a training set of samples comprising profiles of the metabolites that are differentially expressed in Gleason score 6 versus Gleason score 8 prostate tumors.

7. The method of claim 6, wherein training the classifier comprises using a cross-validation technique.

8-9. (canceled)

10. The method of claim 1, wherein the biological sample is selected from the group consisting of blood, serum, urine, and tissue.

11-13. (canceled)

14. A method to detect the presence of high grade prostate tumors in a subject with a Gleason score 7 prostate tumor, the method comprising:
obtaining a biological sample of a subject in need thereof;
measuring a profile of metabolites in the biological sample, wherein the metabolites are differentially expressed in Gleason score 6 versus Gleason score 8 prostate tumors; and
analyzing the profile of the metabolites with at least one processor programmed to implement a specific prediction model to assign a Gleason grade to the sample based on the profile of the metabolites.

15. The method of claim 14, wherein the assigned Gleason grade is 6 or 8.

16. The method of claim 14, wherein the differentially expressed metabolites are selected using a criteria of false discovery rate <0.2.

17. (canceled)

18. The method of claim 14, wherein the specific prediction model comprises a trained classifier.

19-22. (canceled)

23. The method of claim 14, wherein the biological sample is selected from the group consisting of blood, serum, urine, and tissue.

24-26. (canceled)

27. A method to supplement Gleason score evaluation of a Gleason 7 prostate tumor, the method comprising:
classifying, with at least one processor, a profile of a set of metabolites in a biological sample obtained from a subject with a Gleason score 7 prostate tumor to assign a Gleason grade to the sample based on the profile of metabolites, wherein: metabolites in the set of metabolites are differentially expressed in Gleason score 6 versus Gleason score 8 prostate tumors.

28. The method of claim 27, wherein the assigned Gleason grade is 6 or 8.

29. The method of claim 27, wherein the differentially expressed metabolites are selected using a criteria of false discovery rate <0.2.

30. (canceled)

31. The method of claim 27, wherein the profile of metabolites is classified using a trained classifier.

32-41. (canceled)

42. A method, comprising:
training, with at least one processor, a classifier to provide a trained classifier that distinguishes between Gleason score 6 and 8, wherein the classifier is trained using a training set of samples comprising profiles of the metabolites that are differentially expressed in Gleason score 6 versus Gleason score 8 prostate tumors.

43. The method of claim 42, wherein the differentially expressed metabolites are selected using a criteria of false discovery rate <0.2.

44. The method of claim 42, wherein the classifier is trained using a cross-validation technique.

45-47. (canceled)

48. A computer-readable storage medium encoded with a plurality of instructions that, when executed by at least one processor, performs a method comprising:
classifying a profile of a set of metabolites in a biological sample obtained from a subject with a Gleason score 7 prostate tumor to assign a Gleason grade to the sample based on the profile of metabolites, wherein:
metabolites in the set of metabolites are differentially expressed in Gleason score 6 versus Gleason score 8 prostate tumors.

49-50. (canceled)
